(19) United States
(12) Patent Application Publication
(10) Pub. No.: US 2005/0153370 A1
(43) Pub. Date: Jul. 14, 2005

(54) Method of enhancing signal detection of cell-wall components of cells

(75) Inventors: Brinda B. Lakshmi, Woodbury, MN (US); Patrick A. Mach, Shorewood, MN (US); Larry G. Martin, Golden Valley, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/015,166

(22) Filed: Dec. 17, 2004

Related U.S. Application Data
(60) Provisional application No. 60/533,171, filed on Dec. 30, 2003.

(51) Int. Cl. G01N 33/53; G01N 33/567

(52) U.S. Cl. 435/7.2

(57) ABSTRACT

The invention relates to methods of enhancing signal detection of components of cell walls, wherein the methods involve lysing cells to form cell-wall fragments and analyzing the cell-wall fragments.

### CROSS-REFERENCE TO RELATED APPLICATIONS

The present invention claims priority to U.S. patent application Ser. No. 60/533,171, filed on December 30, 2003, which is incorporated herein by reference.

### BACKGROUND

The emergence of bacteria having resistance to commonly used antibiotics is an increasing problem with serious implications for the treatment of infected individuals. Accordingly, it is of increasing importance to determine the presence of such bacteria at an early stage and in a relatively rapid manner to gain better control over such bacteria. This also applies to a variety of other microbes.

One such microbe of significant interest is Staphylococcus aureus (“S. aureus”). This is a pathogen causing a wide spectrum of infections including: superficial lesions such as small skin abscesses and wound infections; systemic and life threatening conditions such as endocarditis, pneumonia and septicemia; as well as toxinoses such as food poisoning and toxic shock syndrome. Some strains (e.g., Methicillin-Resistant S. aureus) are resistant to all but a few select antibiotics.

Current techniques for the detection of microbes, particularly bacteria resistant to antibiotics, are generally time consuming and typically involve culturing the bacteria in pure form. One such technique for the identification of pathogenic staphylococci associated with acute infection, i.e., S. aureus in humans and animals and S. intermedius and S. hyicus in animals, is based on the microbe's ability to clot plasma. At least two different coagulase tests have been described: a tube test for free coagulase and a slide test for bound coagulase or clumping factor. The tube coagulase test typically involves mixing an overnight culture in brain heart infusion broth with reconstituted plasma, incubating the mixture for 4 hours and observing the tube for clot formation by slowly tilting the tube for clot formation. Incubation of the test overnight has been recommended for S. aureus since a small number of strains may require longer than 4 hours for clot formation. The slide coagulase test is typically faster and more economical; however, 10% to 15% of S. aureus strains may yield a negative result, which requires that the isolate by reexamined by the test tube test.

Although methods of detecting S. aureus, as well as other microbes, have been described in the art, there would be advantage in improved methods of detection.

### SUMMARY

The invention provides methods of enhancing signal detection of components of cell walls, wherein the methods involve lysing cells to form cell-wall fragments and analyzing the cell-wall fragments for a component of interest. In particular, the methods are useful for detecting one or more components of cell walls that are characteristic of a microbe, particularly Staphylococcus aureus.

In one embodiment, the present invention provides a method of enhancing signal detection of a cell-wall component of cells. The method includes: providing a test sample including cells; lysing the cells to form a lysate including cell-wall fragments; and analyzing the cell-wall fragments for a cell-wall component; wherein the cell-wall component displays an enhanced signal relative to the same component in unlysed cells.

In another embodiment, a method is provided for enhancing signal detection of a cell-wall component of cells characteristic of Staphylococcus aureus. The method includes: providing a test sample including uncultured cells; lysing the uncultured cells to form a lysate including cell-wall fragments; and analyzing the cell-wall fragments for a cell-wall component characteristic of Staphylococcus aureus; wherein the cell-wall component characteristic of Staphylococcus aureus displays an enhanced signal relative to the same component in unlysed cells.

In another embodiment, a method is provided for enhancing signal detection of a cell-wall component of cells characteristic of Staphylococcus aureus. The method includes: providing a test sample including uncultured cells; contacting the uncultured cells with lysostaphin to form a lysate including cell-wall fragments; and analyzing the cell-wall fragments for protein A; wherein the protein A in the cell-wall fragments displays an enhanced signal relative to the protein A in the cell walls of unlysed cells.

The terms “comprises” and variations thereof do not have a limiting meaning where these terms appear in the description and claims.

As used herein, “a,” “an,” “the,” “at least one,” and “one or more” are used interchangeably.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention provides methods of enhancing signal detection of components of cell walls of cells from prokaryotic and eukaryotic organisms, for example. Such methods involve lysing cells (which may be cultured or uncultured) in a test sample to form cell-wall fragments and analyzing the cell-wall fragments for a component of interest.

In particular, the methods of the present invention are useful for detecting one or more components of cell walls that are characteristic of a species of interest (e.g., a microbe of interest), and optionally one or more internal cell components that are further characteristic of a species of interest (e.g., an antibiotic resistant microbe of interest). Herein, it is believed that the cell-wall fragments analyzed are solid pieces of cell wall. That is, it is believed that they are not solubilized upon lysing; rather, the cell wall is merely broken into pieces. Furthermore, the cell-wall component that is analyzed is still part of (i.e., in or on) the cell wall fragments. That is, they are not solublized upon lysing. Significantly, this enhances the signal of the cell-wall component relative to the same component in an unlysed cell.

Microbes (i.e., microorganisms) of particular interest include Gram positive bacteria, Gram negative bacteria, fungi, protozoa, mycoplasma, yeast, viruses, and even lipid-enveloped viruses. Particularly relevant organisms include members of the family Enterobacteriaceae, or genera Staphylococcus spp., Streptococcus spp., Pseudomonas spp., Enterococcus spp., Esherichia spp., Bacillus spp., Listeria spp., Vibrio spp., as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Vibrio cholera, V. parahemolyticus, Salmonella cholerasuis, S. typhi, S. typhimurium, Candida albicans, C. glabrata, C. krusei, and multiple drug resistant Gram negative rods (MDR).

Gram positive and Gram negative bacteria are of interest. Of particular interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein.

The present invention is advantageous over conventional techniques for analyzing samples for such microbes because the signal for the cell-wall component characteristic of the microbe is enhanced. Such cell-wall components include, for example, cell-wall proteins such as protein A and microbial surface components recognizing adhesive matrix molecules (MSCRAMMs) such as fibrinogen-binding proteins (e.g., clumping factors), fibronectin-binding proteins, collagen-binding proteins, heparin/heparin-related polysaccharides binding proteins, and the like. Protein A and clumping factors, such as fibrinogen-binding factors and clumping factors A, B, and Efb, are also particularly useful in methods of detecting the presence of Staphylococcus aureus. Other cell-wall components of interest include capsular polysaccharides and cell-wall carbohydrates (e.g., teichoic acid and lipoteichoic acid).

Such microbes or other species of interest can be analyzed in a test sample that may be derived from any source, such as a physiological fluid, e.g., blood, saliva, ocular lens fluid, synovial fluid, cerebral spinal fluid, pus, sweat, exudate, urine, mucous, lactation milk, or the like. Further, the test sample may be derived from a body site, e.g., wound, skin, nares, scalp, nails, etc.

The art describes various patient sampling techniques for the detection of microbes such as S. aureus. Such sampling techniques are suitable for the method of the present invention as well. It is common to obtain a sample from wiping the nares of a patient. A particularly preferred sampling technique includes the subject's (e.g., patient's) anterior nares swabbed with a sterile swab or sampling device. For example, one swab is used to sample each subject, i.e., one swab for both nares. The sampling can be performed, for example, by inserting the swab (such as that commercially available from Puritan, East Grinstead, UK under the trade designation “Pure-Wraps”) dry or pre-moistened with an appropriate solution into the anterior tip of the subject's nares and rotating the swab for two complete revolutions along the nares' mucosal surface. The swab is typically then cultured directly or extracted with an appropriate solution typically including water optionally in combination with a buffer and at least one surfactant.

Besides physiological fluids, other test samples may include other liquids as well as solid(s) dissolved in a liquid medium. Samples of interest may include process streams, water, soil, plants or other vegetation, air, (e.g., contaminated) surfaces, and the like.

The test sample (e.g., liquid) may be subjected to prior treatment, such as dilution of viscous fluids. The test sample (e.g., liquid) may be subjected to other methods of treatment prior to injection into the sample port such as concentration, by filtration, centrifugation, distillation, dialysis, or the like; dilution, filtration, inactivation of natural components, addition of reagents, chemical treatment, etc.

This signal enhancement of the cell-wall components occurs as a result of lysing the cells in the test sample. In the methods of the present invention, lysing can include contacting the cells with a lysing agent or physically lysing the cells. Lysing can be conducted under conventional conditions, such as, for example, at a temperature of about 5° C. to about 37° C., preferably at a temperature of about 15° C. to about 25° C. Significantly, the lysing can occur using uncultured cells, i.e., a direct test sample, although cultured cells can be used as well.

As a result of lysing the cells to form cell-wall fragments and the resultant enhancement of the signal of cell-wall components, samples having relatively low concentrations of the species of interest can be evaluated. Thus, advantageously, methods of the invention have improved sensitivity. For example, for certain embodiments, the test sample may include a relatively low concentration of microbes, particularly Staphylococcus aureus. Such relatively low concentrations include, for example, less than about 5×10⁴ colony forming units (“cfu”) per milliliter (cfu/ml) of microbe, less than about 5×10³ cfu/ml, less than about 1000 cfu/ml, and even as low as about 500 cfu/ml. Microbes, such as S. aureus, can be detected at high levels as well, ranging up to as much as 5×10⁷ cfu/ml, for example.

Suitable lysing agents include, for example, enzymes such as lysostaphin, lysozyme, endopeptidases, N-acetylmuramyl-L-alanine amidase, endo-beta-N-acethylglucosaminidase, and ALE-1. Various combinations of enzymes can be used if desired. Lysostaphin is particularly useful in methods of detecting the presence of Staphylococcus aureus.

Other lysing agents include salts (e.g., chaotrophic salts), solubilizing agents (e.g., detergents), reducing agents (e.g., DTT, DTE, cysteine, N-acetyl cysteine), acids (e.g., HCl), bases (e.g., NaOH). Various combinations of such lysing agents can be used if desired.

Lysing can also occur upon physically lysing the cells. Physical lysing can occur upon vortexing the test sample with glass beads, sonicating, boiling, or subjecting the test sample to high pressure, such as occurs upon using a French press.

If desired, methods of the present invention can further include analyzing the lysate for an internal cell component, which may or may not be associated with a cell membrane. Internal cell components are particularly useful in analyzing antibiotic resistant microbes, such as MRSA, VRSA, VISA, VRE, and MDR. Internal cell components that can be characteristic of such microbes include membrane proteins. Examples of such membrane proteins include cytoplasmic membrane proteins, outer membrane proteins, and cell membrane proteins. Cytoplasmic membrane proteins, such as penicillin binding proteins (PBP) (e.g., PBP2′ or PBP2a) can be particularly characteristic of antibiotic resistant microbes. For example, the cytoplasmic membrane protein PBP2′ is characteristic of MRSA.

The methods of the present invention can involve not only detecting the presence of a cell-wall component, but preferably identifying such cell-wall component, which can lead to identifying a microbe for which the cell-wall component is characteristic. In certain embodiments, analyzing the cell-wall fragments for a cell-wall component includes quantifying the cell-wall component.

Depending on the techniques of analyzing used in the methods of the present invention, relatively small volumes of test sample can be used. Although test sample volume as high as 1-2 milliliters (ml) may be utilized, advantageously test samples on the order of 50 microliters (μl) are sufficient for certain methods.

Depending on the techniques of analyzing used in the methods of the present invention, the detection time can be relatively short. For example, the detection time can be less than about 300 minutes, less than about 250 minutes, less than about 200 minutes, less than about 150 minutes, less than about 100 minutes, less than about 60 minutes, and even as short as about 30 minutes.

Such techniques of analyzing can be any of a wide variety of conventional techniques known to one of skill in the art. For example, such methods can include the use of fluorometric immunochromatography (e.g., rapid analyte measurement procedure such as that described in U.S. Pat. No. 5,753,517), acoustic wave sensors, ELISA (e.g., colorimetric ELISA), and other colorimetric techniques (e.g., colorimetric sensors including polydiacetylene (PDA) materials) such as those described in U.S. Patent Application Publication No. 2004/0132217; U.S. patent application Ser. No. 10/325,276, filed Dec. 19, 2002; and Applicants' Assignee's Copending application Ser. No. ______, filed on even date herewith entitled “Colorimetric Sensors Constructed of Diacetylene Materials” (Attorney Docket No. 60422US002), as well as surface plasmon resonance (SPR, using biosensors of the type available from Biacore, Upsala, Sweden).

Enzyme-Linked ImmunoSorbent Assays (ELISA) are based on two important biological phenomena: 1) the discriminatory power of antibodies to differentiate between a virtually limitless number of specific foreign compounds and 2) the ability of enzymes to specifically catalyze detectable chemical reactions. This combination of bound and soluble antibodies' reactions to foreign compounds, along with the detection of these reactions through a subsequent reaction catalyzed by an enzyme attached to the soluble antibody, provide for very sensitive and specific measurements of the foreign compounds. Such techniques are well-known to one of skill in the art.

Surface Plasmon Resonance (SPR) is an optical technique based on surface plasmon resonance that measures changes in refractive index near the surface of the sensor. When light travels from an optically denser medium (i.e., one having a higher refractive index) to a less dense medium (i.e., one having a lower refractive index), total internal reflection (TIR) occurs at the interface between the two media if the angle at which the light meets the interface is above a critical angle. When TIR occurs, an electromagnetic “evanescent wave” propagates away from the interface into the lower refractive index medium. If the interface is coated with a thin layer of certain conducting materials (e.g., gold or silver), the evanescent wave may couple with free electron constellations, called surface plasmons, at the conductor surface. Such a resonant coupling occurs at a specific angle of the incident light, absorbing the light energy and causing a characteristic drop in the reflected light intensity at that angle. The surface electromagnetic wave creates a second evanescent wave with an enhanced electric field penetrating into the less dense medium. The resonance angle is sensitive to a number of factors including the wavelength of the incident light and the nature and the thickness of the conducting film. Most importantly, however, the angle depends on the refractive index of the medium into which the evanescent wave of the surface plasmon wave propagates. When other factors are kept constant, the resonance angle is thus a direct measure of the refractive index of the less dense medium, the angle being very sensitive to refractive index changes in the medium. The SPR evanescent wave decays exponentially with distance from the interface, and effectively penetrates the lower refractive index medium to a depth of approximately one wavelength. Therefore, only changes in refractive index very close to the interface may be detected. This technique can be carried out using using biosensors of the type available from Biacore, Upsala, Sweden.

In certain embodiments of the present invention, a method of analyzing a cell-wall component can involve detecting the change in at least one physical property. This can include a change in viscosity and/or a change in mass that results in a change in wave phase and or wave velocity. In certain embodiments such a change can be detected by a biosensor.

As used herein “biosensor” refers to a device that detects a change in at least one physical property and produces a signal in response to the detectable change. The means by which the biosensor detects a change may include electrochemical means, optical means, electro-optical means, acoustic mechanical means, etc. For example, electrochemical biosensors utilize potentiometric and amperometric measurements, whereas optical biosensors utilize absorbance, fluorescence, visible detection, or luminescence and evanescent waves. For certain embodiments, a biosensor that employs an acoustic mechanical means for detection, such as a surface acoustic wave (SAW) biosensor, can be used. Biosensors employing acoustic mechanical means and components of such biosensors are described, for example, in U.S. Pat. Nos. 5,076,094; 5,117,146; 5,235,235; 5,151,110; 5,763,283; 5,814,525; 5,836,203; and 6,232,139.

Piezoelectric-based SAW biosensors typically operate on the basis of their ability to detect minute changes in mass or viscosity. As described in, e.g., U.S. Pat. No. 5,814,525 (Renschler et al.), the class of piezoeletrfic-based acoustic mechanical devices can be further subdivided into surface acoustic wave (SAW), acoustic plate mode (APM), or quartz crystal microbalance (QCM) devices depending on their mode of detection. APM devices operate on a similar principle to SAW devices, except that the acoustic wave used can be operated with the device in contact with a liquid. Similarly, an alternating voltage applied to the two opposite electrodes on a QCM (typically AT-cut quartz) device induces a thickness shear wave mode whose resonance frequency changes in proportion to mass changes in a coating material.

The direction of the acoustic wave propagation (e.g., in a plane parallel to the waveguide or perpendicular to the plane of the waveguide) may be determined by the crystal-cut of the piezoelectric material from which the biosensor is constructed. SAW biosensors in which the majority of the acoustic wave propagates in and out of the plane (e.g., Rayleigh wave, most Lamb-waves) are typically not employed in liquid sensing applications because of acoustic damping from the liquid in contact with the surface.

For liquid sample mediums, a shear horizontal surface acoustic wave biosensor (SH-SAW) may preferably be used. SH-SAW sensors are typically constructed from a piezoelectric material with a crystal-cut and orientation that allows the wave propagation to be rotated to a shear horizontal mode, i.e., parallel to the plane defined by the waveguide, resulting in reduced acoustic damping loss to a liquid in contact with the detection surface. Shear horizontal acoustic waves may include, e.g., thickness shear modes (TSM), acoustic plate modes (APM), surface skimming bulk waves (SSBW), Love-waves, leaky acoustic waves (LSAW), and Bleustein-Gulyaev (BG) waves.

In particular, Love wave sensors may include a substrate supporting a SH wave mode such as SSBW of ST quartz or the leaky wave of 36° YXLiTaO₃. These modes may preferably be converted into a Love-wave mode by application of thin acoustic guiding layer or waveguide. These waves are frequency dependent and can be generated if the shear wave velocity of the waveguide layer is lower than that of the piezoelectric substrate.

Waveguide materials may preferably be materials that exhibit one or more of the following properties: low acoustic losses, low electrical conductivity, robustness and stability in water and aqueous solutions, relatively low acoustic velocities, hydrophobicity, higher molecular weights, highly cross-linked, etc. In one example, SiO₂ has been used as an acoustic waveguide layer on a quartz substrate. Examples of other thermoplastic and crosslinked polymeric waveguide materials include, e.g., epoxy, polymethylmethacrylate, phenolic resin (e.g., NOVALAC), polyimide, polystyrene, etc. Other potentially suitable waveguide materials and constructions for use with acousto-mechanical sensors used in the detection cartridges of the present invention may be described in, e.g., Applicants' Assignee's PCT Application No. ______, filed on even date herewith, entitled “Acoustic Sensors and Methods” (Attorney Docket No. 60209W0003).

Alternatively, QCM devices can also be used with liquid sample mediums. Biosensors employing acousto-mechanical devices and components may be described in. e.g., U.S. Pat. Nos. 5,076,094 (Frye et al.); U.S. Pat. No. 5,117,146 (Martin et al.); U.S. Pat. No. 5,235,235 (Martin et al.); U.S. Pat. No. 5,151,110 (Bein et al.); U.S. Pat. No. 5,763,283 (Cernosek et al.); U.S. Pat. No. 5,814,525 (Renschler et al.); U.S. Pat. No. 5,836,203 ((Martin et al.); and U.S. Pat. No. 6,232,139 (Casalnuovo et al.). Shear horizontal SAW devices can be obtained from various manufacturers such as Sandia Corporation, Albuquerque, N. Mex. Some SH-SAW biosensors that may be used in connection with the present invention may also described in Branch et al., “Low-level detection of a Bacillus anthracis simulant using Love-wave biosensors on 36° YX LiTaO₃,” Biosensors and Bioelectronics, 19, 849-859 (2004).

As discussed herein, the methods of the present invention may be used in various detection systems and components (such as detection cartridges including biosensors), which may be found in, e.g., U.S. patent application Ser. No. 60/533,169, filed Dec. 30, 2003; PCT Application No. ______ entitled “Acousto-Mechanical Detection Systems and Methods of Use,” filed on even date herewith (Attorney Docket No. 59468W0003); and PCT Application No. entitled “Detection Cartridges, Modules, Systems, and Methods,” filed on even date herewith (Attorney Docket No. 60342WO003).

In some embodiments, the biosensor comprises a reactant (e.g., antibody) that attaches an S. aureus biomolecule of interest to the surface of a piezoelectric biosensor. If S. aureus is present, the lysed cells in the test sample are analyzed for protein A, which is characteristic for S. aureus and can be detected with a protein A specific antibody immobilized on the biosensor surface.

Additionally, lysed cells, such as S. aureus bacteria, release protein markers from the internal portion of the cells (as opposed to the cell-wall portion of the cells). Such protein markers can be detected by an S. aureus reactant molecule. This technique is particularly suitable for detecting methicillin resistant S. aureus (MRSA). In some embodiments, an S. aureus antibody is employed as the S. aureus reactant. “S. aureus antibody” refers to an immunoglobulin having the capacity to specifically bind a given antigen inclusive of antigen binding fragments thereof. The term “antibody” is intended to include whole antibodies of any isotype (IgG, IgA, IgM, IgE, etc.), and fragments thereof from vertebrate, e.g., mammalian species which are also specifically reactive with foreign compounds, e.g., proteins.

Antibodies can be fragmented using conventional techniques and the fragments screened for utility in the same manner as whole antibodies. Thus, the term includes segments of proteolytically cleaved or recombinantly prepared portions of an antibody molecule that are capable of selectively reacting with a certain protein. Non-limiting examples of such proteolytic and/or recombinant fragments include Fab, F(ab′)2, Fab, Fv, and single chain antibodies (scFv) containing a VL and/or VH domain joined by a peptide linker. The scFv's can be covalently or non-covalently linked to form antibodies having two or more binding sites. Antibodies can be labeled with any detectable moieties known to one skilled in the art. In some aspects, the antibody that binds to an analyte one wishes to measure (the primary antibody) is not labeled, but is instead detected indirectly by binding of a labeled secondary antibody or other reagent that specifically binds to the primary antibody.

Various S. aureus antibodies are known in the art. For example, S. aureus antibodies are commercially available from Sigma-Aldrich and Accurate Chemical. Further, S. aureus antibodies are described in U.S. Pat. No. 4,902,616. Typically, the concentration of antibody employed is at least 2 nanograms/ml. Preferably, the concentration of antibody is at least 100 nanograms/ml. For example, a concentration of 50 micrograms/ml can be employed. Typically, no more than about 500 micrograms/ml are employed. As previously described, it is preferred to immobilize the S. aureus antibody on the surface of the biosensor.

One or more of the analysis techniques described herein can be coupled with electrical and/or electrochemical methods. Microbial metabolism usually results in an increase in both conductance and capacitance causing decrease in impedance. Therefore measurements pertaining to these concepts have been used in the literature to detect bacteria. For example, a re-usable Bulk acoustic wave impedance sensor has been developed for detection of micro-organisms. These organisms are able to transduce their metabolic redox reactions into quantifiable electrical signals. Therefore electrochemical methods have also been used to detect the bacterial organisms. The methods include direct potentiometric detection, light-assisted potentiometric sensing (LAPS), and amperometric detection. An ELISA technique coupled with oxidation-reduction reaction with horseradish peroxide tagged antibody has been monitored electrochemically. Other variations include immunofiltration techniques combined with amperometric sensing. Such techniques are described in D. Ivinitski et al., Biosensors & Bioelectronics, 14, 599-624 (1999).

### EXAMPLES

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

### Example 1

### ELISA Detection

Preparing the Plates with Antibody

Polystyrene microwell plates (Costar 96 Well Cell Culture Cluster, Flat Bottom with Lid, Tissue Culture Treated, Non-pyrogenic, Polystyrene plates, Catalogue number 3596, Corning Incorporated, Corning, N.Y.) were coated with ChromPure Rabbit IgG (whole molecule, Catalog number 011-000-003, Jackson ImmunoResearch Laboratories, West Grove, Pa.) antibody at 10 micrograms/milliliter. The antibody solution was prepared by diluting the antibody in 0.1 M Sodium Bicarbonate, pH 9.6 (Sigma-Aldrich, St. Louis, Mo.). The coated plates were incubated at 37° C. for one hour.

Washing the Plates

The plates were then washed by aspiration and dispensing into each well 0.25 milliliters of a “PBS buffer” solution consisting of 0:02 M Sodium Phosphate (Sigma-Aldrich) and 0.15 M Sodium Chloride (Sigma-Aldrich), to which 0.05% volume-volume (v/v) polyoxyethylene(20) sorbitan monolaurate, (trade designation TWEEN 20 available from, Sigma-Aldrich, St. Louis, Mo.) had been added, the solution pH was 7.5 and the wash was repeated through 5 cycles.

Blocking the Plates

A blotto solution was prepared by mixing Carnation Non-Fat Dry Milk (Nestle USA, Inc., Solon, Ohio) with the wash solution above at a 2% weight by volume (w/v) loading. A portion of this blotto solution (0.2 ml) was added to each well and the plates incubated at 37° C. for 1 hour. The plates were then washed as described above.

Bacteria Suspension Preparation

S. aureus bacteria were obtained from The American Type Culture Collection, Rockville, Md. under the trade designation “ATCC 25923.” The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 rpm for 15 minutes in an Eppendorf model number 5804R centrifuge (Brinkman Instruments, Westbury, N.Y.) and resuspended into PBS buffer containing 0.2% (w/v) PLURONIC L64 Surfactant (BASF Corporation, Mount Olive, N.J.) and washed by centrifugation for 3 additional cycles with this solution.

Bacteria Dilution

The washed bacterial suspensions were then diluted into the following solutions.

Solution 1 was PBS buffer with 0.2% (w/v) PLURONIC L64 Surfactant (BASF Corporation).

Solution 2 was a buffer made by combining 0.01 M Tris-HCL, 1 mM EGTA, 1% Triton X-100, 2.5 mM sodium pyrophosphate, 1 mM Sodium Phosphate, and 1 μg/ml leupeptin (Sigma-Aldrich, St. Louis, Mo.).

Solution 3 was lysing buffer made by combining Solution 2 above with lysostaphin at 3 micrograms/milliliter (catalog number L-4402, Sigma-Aldrich).

S. aureus bacteria were diluted in serial five-fold dilutions to 10⁸, 2×10⁷, 4×10⁶, 8×10⁵, and 1.6×10⁴/milliliter into each of the three solutions.

Cultures of S. epidermidis ATCC 12228 (American Type Culture Collection, Rockville, Md.) were prepared in the same manner and the S. epidermidis bacteria was resuspended only into solution 3 at 10⁸/milliliter as a comparative.

ELISA Testing of Antigen Solutions

Samples of each antigen preparation and dilution as well as samples of each solution containing no bacteria were added to the previously coated, blocked, and washed plates. Each sample was plated in duplicate by adding 0.1 ml of the sample solution into separate microwells on the plate. Plates were incubated at 37° C. for 1 hour. The plates were then washed as above and 0.1 ml of a primary antibody solution added to the appropriate wells.

The primary antibodies were biotinylated Rabbit-anti-S. aureus IgG (Biotin Rabbit Anti-Staphylococcus aureus, Catalog number YVS6887, Accurate Chemical and Scientific Company, Westbury, N.Y.) and biotinylated Mouse anti-Protein A IgG (Monoclonal Anti-Protein A Clone SPA-27, Biotin Conjugate, Catalog number B-3150, Sigma-Aldrich, St. Louis, Mo.). These antibodies were diluted to 5 micrograms/milliliter in blotto and 0.1 milliliter of a primary antibodies solution was added to the appropriate wells. Plates were incubated at 37° C. for 1 hour.

After incubation, the plates were washed as above and 0.1 milliliter of Streptavidin-alkaline phosphatase conjugate (SA-AP, Jackson ImmunoResearch Laboratories) preparation was added to the appropriate wells. Streptavidin-alkaline phosphatase conjugate (SA-AP) preparation was made by diluting Streptavidin-alkaline phosphatase conjugate (Catalog number 016-050-084, Jackson ImmuoResearch Laboratories) to 0.5 microgram/milliliter in blotto. Plates were incubated at 37° C. for 1 hour and then washed as above.

After washing, a 0.1 milliliter portion of an alkaline phosphatase substrate preparation was added to the appropriate wells. The alkaline phosphatase substrate preparation was para-nitrophenyl phosphate substrate (pNPP, Product code 50-80-00, Kirkegaard and Perry Laboratories, Gaithersburg, Md.) prepared per manufacturers instruction. The plates were then incubated at room temperature for 15 minutes. After the 15-minute incubation period, 0.1 milliliter of 5% (w/v) disodium EDTA (Siga-Aldrich) were added to stop the enzyme catalyzed substrate development.

Plates were read with a Bio-Tek Model EL808 Microwell plate reader (Bio-Tek Instruments, Inc., Winooski, Vt.) at 405 nanometers and the results are in Table 1 below (N/A=not applicable (i.e., not measured)).

TABLE 1
ELISA Results
(Absorbance at 405 nm)

| Primary | | Bacteria Concentration in cfu/ml | | | | | |
|---|---|---|---|---|---|---|---|
| Antibody | Solution | 10⁸ | 2 × 10⁷ | 4 × 10⁶ | 8 × 10⁵ | 1.6 × 10⁵ | Buffer |
| Rabbit-Biotin | PBS-L64 Buffer | 2.730 | 1.107 | 0.376 | 0.192 | 0.192 | 0.267 |
| Rabbit-Biotin | Unlysed S. aureus | 2.126 | 0.679 | 0.235 | 0.163 | 0.534 | 0.144 |
| Rabbit-Biotin | Lysed S. aureus | 4.000 | 4.000 | 4.000 | 4.000 | 1.321 | 0.162 |
| Rabbit-Biotin | Lysed S. epidermidis | 0.300 | N/A | N/A | N/A | N/A | 0.134 |
| Mouse-Biotin | PBS-L64 Buffer | 3.895 | 1.322 | 0.409 | 0.243 | 0.157 | 0.166 |
| Mouse-Biotin | Unlysed S. aureus | 4.000 | 1.246 | 0.371 | 0.265 | Na | 0.136 |
| Mouse-Biotin | Lysed S. aureus | 4.000 | 4.000 | 4.000 | 4.000 | 4.000 | 0.194 |
| Mouse-Biotin | Lysed S. epidermidis | 0.715 | N/A | N/A | N/A | N/A | 0.267 |

### Example 2

### Fluorescent Assay Detection

Bacteria Suspension Preparation and Dilution

S. aureus bacteria were obtained from The American Type Culture Collection, Rockville, Md. under the trade designation “ATCC 25923.” The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 revolutions per minute (rpm)) for 15 minutes in an Eppendorf model number 5804R centrifuge (Brinkman Instruments, Westbury, N.Y.) and resuspended into PBS buffer with 0.2% weight by volume (w/v) PLURONIC L64 Surfactant (BASF Corporation, Mount Olive, N.J.) and washed by centrifugation for 3 additional cycles with this solution.

The washed S. aureus 25923 suspension was then diluted in 10-fold serial dilutions from 10⁵ to 10³/milliter into two different diluents (E5 to E3). The first was RAMP Assay Sample Buffer No. 1 (Response Biomedical Corporation, Burnaby, BC, Canada) and the second was the same as the first buffer only lysostaphin (Sigma-Aldrich) was added to give 3 micrograms/milliliter solution. Samples of buffer alone were also run (E0).

Assays were performed on a RAMP fluorescent assay reader (Response Biomedical Corporation, Burnaby, BC, Canada) following the Manufacturer's directions. The results are given below in Table 2.

TABLE 2
RAMP Testing with Whole and Lysed S. aureus 25923

| Sample Concentration | Whole Cells - S. aureus | Lysed S. aureus 25923 |
|---|---|---|
| (cfu/ml) | 25923 (dUnits) | (dUnits) |
| E5 | 51.4 | 999 |
| E4 | 55.7 | 108.3 |
| E3 | 55.8 | 83.8 |
| E0 | 44.8 | 56.5 |

### Example 3

### Colorimetric Detection

Coating Polydiacetylene Liposomes on a Polycarbonate Membrane

A formulation of (60/40) diacetylene HO(O)C(CH₂)₂C(O)O(CH₂)₄—C≡C—C≡C(CH₂)₄O(O)C(CH₂)₁₂CH₃ (prepared as in Example 6 of U.S. Pat. Application Publication No. 2004/0132217) and 1,2-dimeristoyl-sn-glycero-3-phosphocholine (DMPC, formula weight (F.W.) 678, available from Sigma-Aldrich, catalog number P2663) was coated onto 25 mm diameter porous polycarbonate membranes with 200 nm diameter pores (Avestin, Inc., Ottawa, Canada) to make colorimetric detector samples. The membranes were coated using a handheld extrusion process.

The 60/40 diacetylene/DMPC mixture was weighed into a glass vial and suspended in HEPES buffer (5 mM, pH 7.2) to produce a 1 mM solution. This solution was then probe sonicated using a Misonix XL202 probe sonicator for 2 minutes, and placed in a 4° C. refrigerator for about 20 hours. This process results in the formation of a polydiacetylene (PDA) liposome suspension.

The polycarbonate membrane to be coated was placed into the stainless steel chamber of a handheld extruder system, trade designation LIPOFAST, available from Avestin, Inc. (Ottawa, Canada). The membrane covered the bottom O-ring of the TEFLON base. Care was taken to avoid bending and/or creasing the membrane. The top TEFLON O-ring block was placed inside the stainless steel housing on top of the membrane. The chamber was then sealed by tightening the stainless steel caps by hand. A Gas Tight syringe (Hamilton 500-microliter (μl)) was filled with a suspension of diacetylene liposomes and attached to the base and a second syringe was attached to the other cap. The liposomes of the first syringe were forced slowly through the chamber with constant even pressure.

The membrane captured the liposomes on the surface allowing the clear buffer to flow slowly through and into second syringe. This action was considered a 1 pass coating. The membrane samples used as detectors in this example used 2 passes of coating. The second pass was applied like the first by a second 0.5 milliliter (ml) portion of liposome being applied to the already coated membrane. The second syringe containing the filtered buffer was removed and the contents were discarded. The stainless steel end cap was unscrewed and the TEFLON O-ring block removed. The wet membrane was removed and placed coated side up on a glass slide and placed in a refrigerator at 5° C. for at least 3 hours. The sample was then dried in a dessiccator containing CaSO₄ for 30 minutes and exposed to 254 nanometer (nm) UV light for 30-90 seconds.

The PDA-coated substrate (25 millimeter (mm) circle) was cut into four quarters. Each quarter sample was used as a sample for an experiment. The substrates were placed in separate wells of 24-well microtiter plates. A phosphate buffer saline solution was prepared by diluting ten-fold a 10× PBS liquid concentrate (available commercially from EMD Biosciences, San Diego Calif.). This results in a PBS buffer solution with the following salt composition: 10 mM Sodium Phosphate, 137 mM Sodium Chloride, 2.7 mM Potassium Chloride. To the PBS buffer was also added 0.2% (w/v) PLURONIC L64 surfactant (available commercially from BASF Corporation, Mount Olive, N.J.) yielding a PBS L64 buffer solution. Whole bacteria sample solutions were prepared by mixing 250 μl PBS L64 buffer solution containing whole S. aureus bacteria ATCC 25923 with 250 μl of antibody solution. The antibody solution contained Rabbit anti-Staphylococcus aureus (Catalog number YVS6881, Accurate Chemical and Scientific Corp.) at a concentration of 100 μg/ml in PBS L64 buffer solution. Samples containing lysed S. aureus bacteria ATCC 25923 in PBS L64 buffer solution were prepared using a lysing buffer which consisted of lysostaphin lysostaphin at 3 micrograms/milliliter (catalog number L-4402, Sigma-Aldrich) in PBS L64 buffer solution. Lysed bacteria sample solutions consisted of 250 μl of the lysed S. aureus bacteria ATCC 25923 in PBS-L64 mixed with 250 μl of the antibody solution prepared as described above. The concentration of bacteria used in the test samples varied between 0 and 10⁵ cfu/ml as reported in Table 3 below. The mixture of the bacteria and antibody solution was allowed to stand for 5 minutes and then added onto the 24-well plate containing the PDA-coated substrate. Control samples were also prepared for comparison. The control sample contained no bacteria and consisted simply of 250 μl of PBS-L64 buffer mixed with 250 μl of the antibody solution prepared as described above.

A picture was taken every 5 minutes using a digital camera. The picture was scanned using software from Adobe Systems Incorporated (San Jose, Calif.), trade designation ADOBE PHOTOSHOP version 5.0, to obtain the RGB (Red, Green, Blue) channel values for each sensor. Colorimetric response (CR) was determined using the red and blue channel values as given by the equation CR=((PRᵢₙᵢₜᵢₐₗ−PRₛₐₘₚₗₑ)/PRᵢₙᵢₜᵢₐₗ) where PR=percent red value of the sample, and is given by the equation PR=Rᵥₐₗᵤₑ/(Rᵥₐₗᵤₑ+Bᵥₐₗᵤₑ)*100, where Rᵥₐₗᵤₑ and Bᵥₐₗᵤₑ correspond to the value of the polydiacetylene sensor's red and blue channel respectively. The data in the Table 3 below shows the difference in the colorimetric response between a control sample and the bacteria containing sample (either whole or lysed), measured at 15 minutes.

TABLE 3
Difference in Colorimetric Response

| | Colorimetric Response | |
|---|---|---|
| | Difference from | Colorimetric Response |
| Bacteria | Control for Whole | Difference from Control for |
| Concentration | Bacteria | Lysed Bacteria |
| (cfu/ml) | (Δ Fraction Red) | (Δ Fraction Red) |
| 0 | 0 | 0 |
| 100 | 0.05 | 0.17 |
| 1,000 | 0.05 | 0.58 |
| 10,000 | 0.05 | 0.52 |
| 100,000 | 0.04 | 0.64 |

The complete disclosures of the patents, patent applications, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows

1. A method of enhancing signal detection of a cell-wall component of cells, the method comprising:
providing a test sample comprising cells;
lysing the cells to form a lysate comprising cell-wall fragments; and
analyzing the cell-wall fragments for a cell-wall component;
wherein the cell-wall component displays an enhanced signal relative to the same component in unlysed cells.

2. The method of claim 1 wherein the cell-wall component comprises a cell-wall protein.

3. The method of claim 2 wherein the cell-wall protein is protein A.

4. The method of claim 2 wherein the cell-wall protein is a clumping factor.

5. The method of claim 1 wherein the cell-wall component comprises a capsular polysaccharide or a cell-wall carbohydrate.

6. The method of claim 1 wherein lysing the cells comprises contacting the cells with a lysing agent.

7. The method of claim 6 wherein the lysing agent comprises an enzyme selected from the group consisting of lysostaphin, lysozyme, endopeptidases, N-acetylmuramyl-L-alanine amidase, endo-beta-N-acethylglucosaminidase, ALE-1, and combinations thereof.

8. The method of claim 6 wherein the lysing agent comprises a salt, a solubilizing agent, a reducing agent, an acid, a base, or combinations thereof.

9. The method of claim 1 wherein lysing the cells comprises physically lysing the cells.

10. The method of claim 1 wherein the cells comprise one or more microbes.

11. The method of claim 10 wherein the microbes comprise a gram positive bacteria.

12. The method of claim 11 wherein the gram positive bacteria comprise Staphylococcus aureus.

13. The method of claim 10 wherein the microbes comprise a gram negative bacteria.

14. The method of claim 1 wherein the cells are uncultured.

15. The method of claim 1 wherein the method further comprises analyzing the lysate for an internal cell component.

16. The method of claim 15 wherein the cells comprise antibiotic resistant microbes.

17. The method of claim 15 wherein the internal cell component comprises a cell membrane.

18. The method of claim 17 wherein the cell membrane comprises a membrane protein.

19. The method of claim 18 wherein the membrane protein is a cytoplasmic membrane protein.

20. The method of claim 19 wherein the cytoplasmic membrane protein is PBP2′.

21. The method of claim 1 wherein analyzing the cell-wall fragments for a cell-wall component comprises identifying the cell-wall component.

22. The method of claim 1 wherein analyzing the cell-wall fragments for a cell-wall component comprises quantifying the cell-wall component.

23. The method of claim 1 wherein analyzing the cell-wall fragments for a cell-wall component comprises analyzing with fluorometric immunochromatography.

24. The method of claim 1 wherein analyzing the cell-wall fragments for a cell-wall component comprises analyzing with ELISA.

25. The method of claim 1 wherein analyzing the cell-wall fragments for a cell-wall component comprises analyzing with an acoustic wave sensor.

26. The method of claim 1 wherein analyzing the cell-wall fragments for a cell-wall component comprises analyzing colorimetrically.

27. A method of enhancing signal detection of a cell-wall component of cells characteristic of Staphylococcus aureus, the method comprising:
providing a test sample comprising uncultured cells;
lysing the uncultured cells to form a lysate comprising cell-wall fragments; and
analyzing the cell-wall fragments for a cell-wall component characteristic of Staphylococcus aureus;
wherein the cell-wall component characteristic of Staphylococcus aureus displays an enhanced signal relative to the same component in unlysed cells.

28. The method of claim 27 wherein the cell-wall component comprises a cell-wall protein.

29. The method of claim 28 wherein the cell-wall protein is protein A.

30. The method of claim 27 wherein lysing the uncultured cells comprises contacting the uncultured cells with lysostaphin.

31. The method of claim 27 wherein the method further comprises analyzing the lysate for an internal cell component.

32. The method of claim 31 wherein the internal cell component comprises a cell membrane.

33. The method of claim 32 wherein the cell membrane comprises a membrane protein.

34. The method of claim 33 wherein the membrane protein is a cytoplasmic membrane protein characteristic of MRSA.

35. The method of claim 34 wherein the cytoplasmic membrane protein is PBP2′.

36. The method of claim 27 wherein analyzing the cell-wall fragments for a cell-wall component comprises quantifying the cell-wall component.

37. The method of claim 27 wherein the test sample comprises Staphylococcus aureus at a concentration of less than 5×10⁴ cfu/ml.

38. A method of enhancing signal detection of a cell-wall component of cells characteristic of Staphylococcus aureus, the method comprising:
providing a test sample comprising uncultured cells;
contacting the uncultured cells with lysostaphin to form a lysate comprising cell-wall fragments; and
analyzing the cell-wall fragments for protein A;
wherein the protein A in the cell-wall fragments displays an enhanced signal relative to the protein A in the cell walls of unlysed cells.
